(19) United States
(12) Patent Application Publication
(10) Pub. No.: US 2025/0017584 A1
(43) Pub. Date: Jan. 16, 2025

(54) PROXIMAL BICEPS CLIP

(71) Applicant: Michel Arcand, Chepachet, RI (US)

(72) Inventor: Michel Arcand, Chepachet, RI (US)

(21) Appl. No.: 18/768,319

(22) Filed: Jul. 10, 2024

Related U.S. Application Data
(60) Provisional application No. 63/513,138, filed on Jul. 12, 2023.

(51) Int. Cl. A61B 17/04; A61B 17/00

(52) U.S. Cl. CPC A61B 17/0487; A61B 17/00234; A61B 17/04; A61B 2017/00238; A61B 2017/00407; A61B 2017/00477; A61B 2017/00831

(57) ABSTRACT

The present invention is directed to a proximal biceps clip for the repair of a biceps tendon and method of performing a biceps tenodesis medical procedure using such a clip that has a locking clamp configuration with a tendon grasping groove with teeth thereon. The clip has hinge and a locking portion as well as recesses to receive gripping arms or fingers of an instrument for closure thereof to facilitate the locking and closure step. The present invention allows easier and more precise placement of a clip device that securely holds the tendon into position while the tenodesis process occurs. This saves time, improves the position in which the tenodesis will occur and reduces the overall cost of the procedure. As a result, the muscle length of the long head portion of the biceps tendon is more anatomic.

### CROSS REFERENCE TO RELATED APPLICATION

This patent document claims priority to earlier filed U.S. Provisional Patent Application Ser. No. 63/513,138, filed Jul. 12, 2023, the entire contents of which are incorporated herein by reference.

### BACKGROUND OF THE INVENTION

The present invention relates generally to the field of medical surgical procedures and, more specifically, to the field of biceps tenodesis, which is an orthopedic surgery performed to repair the tendon that connects a bicep muscle to the shoulder. For example, biceps tenodesis is commonly used to address tendon issues in the shoulders that often occur in athletes and other patients. The goal of such biceps tenodesis surgery is to relieve shoulder pain associated with inflammation and tendon wear due to such injury, overuse, or aging.

As in known medicine, there are several ways to perform a biceps tenodesis, otherwise known as bicep tendon surgery. This well-known procedure involves detaching the long head of the biceps tendon from its attachment on the glenoid socket of the shoulder joint. The tendon passes in the joint and therefore has a decreased blood supply making it vulnerable to injury. In open procedures, the joint is opened and the tendon is cut. Sutures are then passed through the tendon. The tendon is then fixed to the humerus or structures attached to the humerus. This typically results in scar forming at the connection of the tendon to the bone. It can be fixed in the bicipital groove or to the pectoralis tendon as well as other structures. Anchors or screws are often used for this purpose. All of these methods require several steps to perform. They usually also require the use of an anchor button or screw.

There have been attempts in the prior art to solve these problems. However, prior art devices do not resemble a clip or clamp structure. For example, Smith and Nephew manufacture an enobutton that can be used to fix the tendon to the bone but a suture is used to secure the biceps tendon and the button is placed into the bone. Arthrex also manufactures a similar button to the Smith and Nephew button. Again a suture is required to secure the tendon and the button is placed in the bone. Arthrex also makes anchors which are used to fix the sutured tendon to the bone.

Therefore, a common problem in biceps tenodesis is to obtain a secure fixation of the tendon. It then needs to be held in position of long enough that it fuses itself to the bone. This can be accomplished several ways. Using a suture to grasp a tendon through an arthroscopic cannula can be challenging. Using another device to fix the tendon requires several steps. This can undesirably take up quite a bit of surgical time. Proper positioning of the device that will hold the tendon can also be challenging. The device needs to be placed and proper tension has to be applied for the construct to result in tenodesis.

There is a need for a clip device that makes it easier to grasp the tendon. A device is also needed to simplify the procedure. During its application the clip can be better positioned on the tendon. This allows the surgeon to forgo using complicated sutures being passed.

There is yet another need for a device clip to hold the tendon for about 8-10 weeks for the tendon to fix itself to the bone. There is a desire for it to be incorporated in the scar that is tenodesing the biceps tendon.

There is also a need for a device with suture holes to be present to allow a device to have a secondary means of fixation in case it is used in either open situations or if there is a compromise of a portion of the restraints present at the entrance to the bicipital groove.

There is a need for the device to use an increased dimension to prevent the biceps to retract down the groove. Any compromise of the soft tissue restraints to the sides and top of the bicipital groove may need secondary fixation.

There is another need for a device to be easy and inexpensive to manufacture as well as 3D printable out of various materials that are biocompatible and have been used in the manufacturing of medical devices.

Thus, there is a need for a device and method that is to be used in arthroscopic shoulder surgery to speed up and make biceps tenodesis easier and more efficient. It could also be used in open surgery. There is a need for a device that has holes in it to allow suture to be passed and allow use in open surgery. There is a need for a device that may also have a use in other areas where round tendons need to be secured.

### SUMMARY OF THE INVENTION

The present invention preserves the advantages of prior art methods for biceps tenodesis and devices used in connection with such procedures. In addition, it provides new advantages not found in currently available devices and methods and overcomes many disadvantages of such currently available devices and methods.

The present invention is a device and associated method that is to be used in arthroscopic shoulder surgery to speed up and make the medical procedure of biceps tenodesis easier, more efficient, and less expensive. Thus, the clip of the present invention has particular use for being deployed inside the joint with the assistance of a scope rather than outside the joint via open surgery. However, the clip of the present invention could also be used in open surgery. The device preferably includes holes in it to allow suture to be passed and allow use in open surgery and for a secondary structure for securing the clip in place, if desired. It may also have a use in other areas where round tendons need to be secured.

The present invention solves the known problems associated with biceps tenodesis, including the need to place a good grasping suture on the tendon, which otherwise would be technically difficult and time consuming. Thus, the clip of the present invention is smaller and sleeker than prior art devices and, as a result, can be deployed into the joint via arthroscopic surgery. For example, the present invention is small enough and configured, i.e. narrow enough, so that it may be pushed into the joint whereby the clip of the present invention can easily slide into the desired portal and location therein. For example, known devices, such as those manufactured by Smith & Nephew, can be used for accessing and inserting the device into such portals.

Further, the present invention provides a clip that has a central clamp capture area that is sized to receive the biceps tendon factoring in the thickness, size and circumference of the portion thereof that is to be captured by the clip of the present invention. Such a configuration permits the clip of the present invention to capture the tendon enough to securely grip it while allowing for the clip to close with the assistance of an arthroscopic gripping tool, such as a Scorpion Suture Passer tool manufactured by Anthrex. More specifically, appropriate opposing notches in the clip body are provided to locate the fingers of the gripping tool for optimum force to quickly and easily close the clip. The clip includes a ratchet-like closure to selectively secure the clip in a closed condition.

Time in an operating room can be very expensive. Where a suture can be placed for optimal positioning is also technically difficult. The device clip of the present invention allows easier and more precise placement of a device that will securely hold the tendon into position while the tenodesis process occurs. This saves time and also improves the position in which the tenodesis will occur. The muscle length of the long head portion of the biceps tendon would be more anatomic.

Similar problems are also encountered during open tenodesis. By removing the need to secure the tendon with suture, a time advantage will be gained. The tendon can also be secured to the bone, if needed, by the suture holes to provide a secondary structure for securing the biceps tendon.

### BRIEF DESCRIPTION OF THE DRAWING FIGURES

The novel features which are characteristic of the present invention are set forth in the appended claims. However, the invention's preferred embodiments, together with further objects and attendant advantages, will be best understood by reference to the following detailed description taken in connection with the accompanying drawings in which:

FIG. 1 shows a perspective view of the clip of the present invention in an open condition;

FIG. 2 shows a top view of the clip of the present invention of FIG. 1 in an open condition;

FIG. 3 shows a top view of the clip of the present invention of FIG. 1 in a closed condition showing a cross-section of a biceps tendon captured therein;

FIG. 4 shows representational anatomy of a biceps tendon and shoulder;

FIG. 5 shows a technique showing an instrument pulling the biceps tendon in for attachment and deployment of the clip;

FIG. 6 shows a first step of a grasper method for deploying the clip of the present invention;

FIG. 7 shows a second step of a grasper method for deploying the clip of the present invention;

FIG. 8 shows a close-up view of a deployed clip of the present invention wedged in the bicipital groove;

FIG. 9 shows the clip of the present invention deployed in the bicipital groove as viewed from the outside of the joint; and

FIG. 10 shows the clip of the present invention deployed in the bicipital groove as viewed from inside the joint.

### DESCRIPTION OF THE INVENTION

In view of the foregoing, the present invention provides a new, improved, and novel clip device 10 to secure a biceps tendon 12 during a biceps tenodesis medical procedure. The ability to provide such a method and clip device 10 is a significant advance over prior art devices and methods because the procedure can be carried out arthroscopically in a faster and simpler manner with superior results.

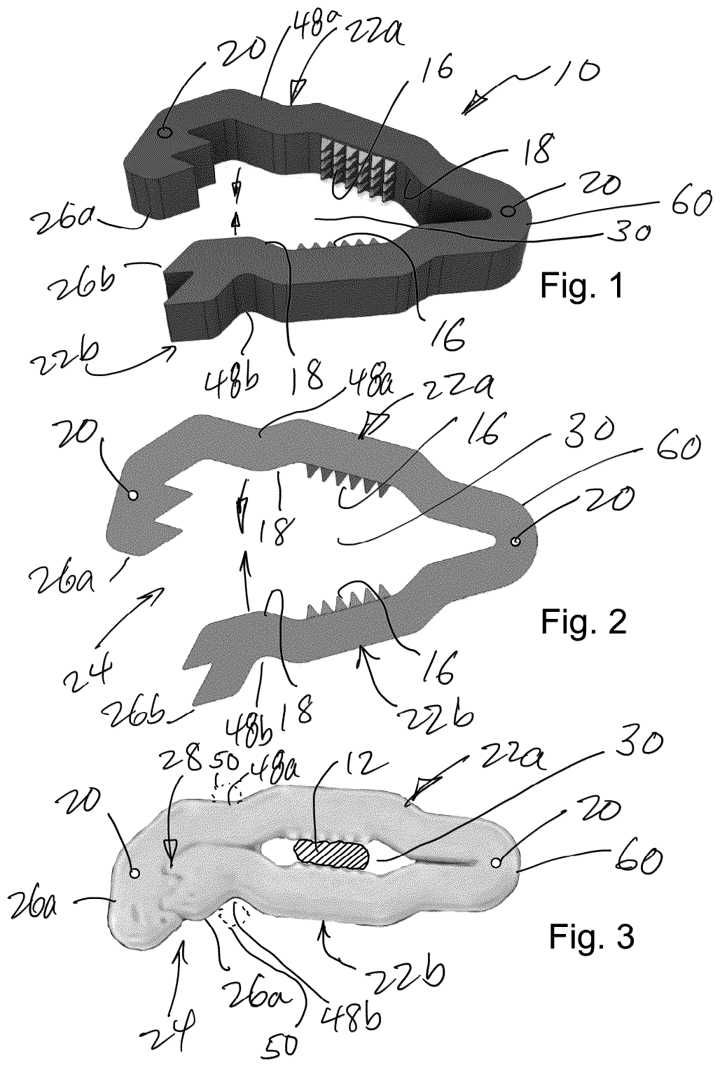

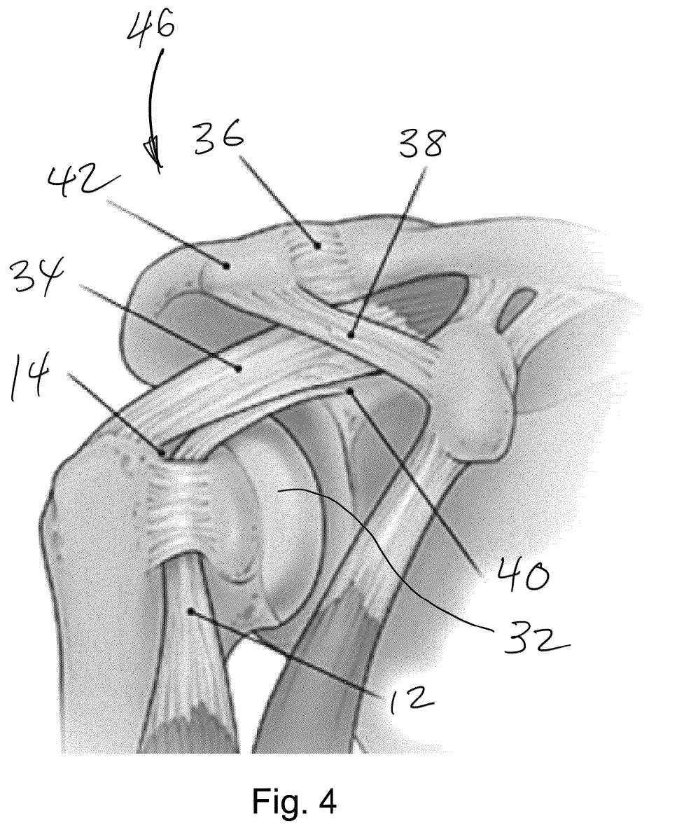

Referring first to FIG. 1, a perspective view of the clip 10 of the present invention in an open condition is shown. The clip device 10 of the present invention is a clamp/clip (hereafter “clip”) for the biceps tendon 12, as shown in FIG. 4, that prevents the proximal tendon 12 to retract down the arm as long as it is larger than the bicipital groove 14 in which the tendon resides. Teeth 16 are provided on the inner surface 18 of the clip 10 to assist in grasping the tendon 12. This simplifies the medical procedure because a suture in the tendon 12 is no longer needed to securely fix itself to the tendon 12. A number of holes 20 through the body of the clip 10 may be provided to enable suturing of the tendon as a secondary securing operation. A first side 22a and a second side 22b are provided.

FIG. 2 shows a top view of the clip 10 of the present invention of FIG. 1 in an open condition. As can be seen, the closure 24 of the clip 10 has a ratchet-like configuration where two opposing sides 22a and 22b of the clip 10 are brought into communication with one other so the respective free ends 26a and 26b engage with one other. The present invention shows a two-step ratchet 28 whereby the amount of ratcheting closure of clip 10 can be selected depending on the size, configuration and circumference of the biceps tendon 12. More or less than two levels of ratcheting may be employed and still be within the scope of the present invention. Also, the dimensions of the clip 10 are preferably 1.5-2 cm in length, 0.5-0.7 cm in width, and 1-1.5 cm in thickness to accommodate most patients but the different sizes of the clip can also be provided.

FIG. 3 shows a top view of the clip 10 of the present invention of FIG. 1 in a closed condition showing a cross-section of a biceps tendon 12 captured therein. As can be seen, the biceps tendon 12 is secured in the clip 10 and, in this case, both ratchets ends 26a and 26b are employed to ratchet the clip 10 down so that it is sufficiently secured in place. An open area 30 is provided to accommodate the size and thickness of the biceps tendon 12 while enabling the clip 10 to be closed enough so at least one of the teeth ratchets 26a and 26b engage. Inner teeth 16 are provided to provide added grip to the biceps tendon 12. Thus, the combined closure force of the ratcheting and the inner teeth 16 fully secure the clip 10 of the present invention to the biceps tendon 12.

Moreover, it would also not necessarily require another device to fix it to the bone. Once the clip 10 of the present invention is in position on the tendon 12, cutting the tendon 12 allows the clip device 10 to wedge itself in the bicipital groove 14 obviating the need for further steps for the procedure, which is a significant advance over known methods and devices.

In a case where the tenodesis is being performed open or where there is a compromise in the soft tissue envelope of the bicipital groove 14, suture holes 20, as in FIGS. 1-3, mentioned above, may be provided that would allow a secondary fixation to uncompromised soft tissue or to the bone.

Turning now to FIG. 4, a representational anatomy of a biceps tendon 12 and shoulder is shown. In FIG. 4, the long head of the biceps tendon 12 can be seen residing in the bicipital groove 14 at the humeral head 32. The supraspinatus tendon 34, acromioclavicular joint 36 and coracoracromial ligament 38, glenoid tubercle 40, and acromion 42 can be seen for context. The clip 10 of the present invention and the methods herein are employed if the biceps tendon 12 is damaged, torn, or the like, and there is a need to repair it.

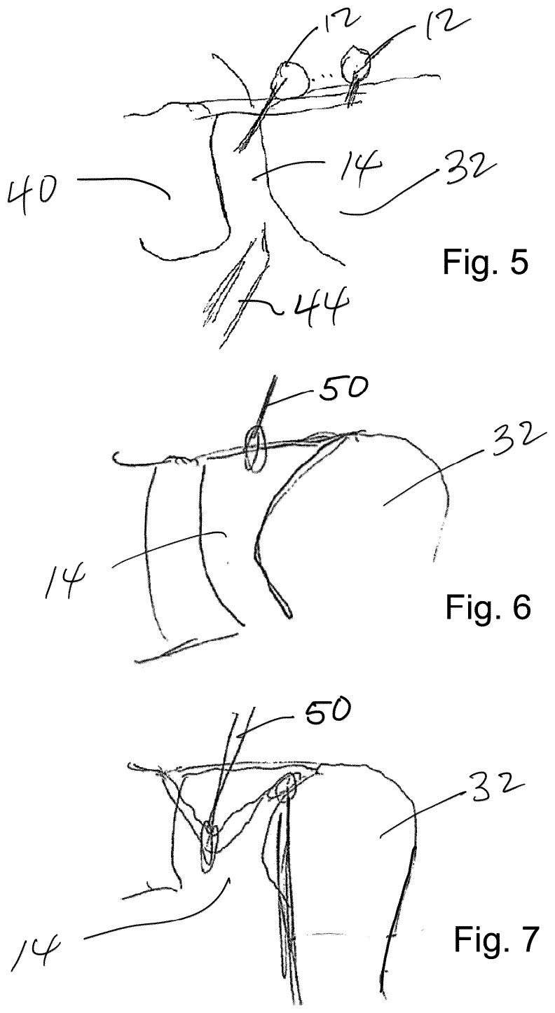

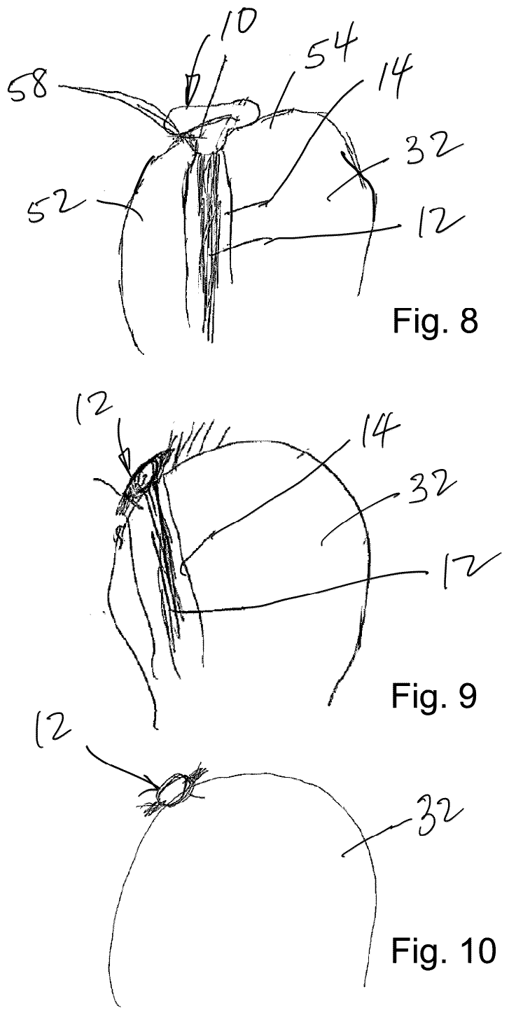

FIGS. 5 to 7 show various method steps to carry out the deployment of the clip 10 of in accordance with the present invention. It should be understood that these are examples of how the clip 10 of the present invention can be deployed in the joint, generally referred to as 46, to secure the biceps tendon 12 in place. For example, FIG. 5 shows a technique showing an instrument pulling the biceps tendon 12 in for attachment and deployment of the clip 10. A scope and camera 44 are representationally shown from below whereby the biceps tendon 12 is pulled through between the glenoid 40 and the head 32 of the joint 46 so that the clip 10 can be connected to the free end of the biceps tendon 12. Preferably, tendon 12 that extends beyond the clip 10 is trimmed. Upon release of the biceps tendon 12, now carrying the clip 10, the biceps tendon 12 withdraws back down the bicipital groove 14 until the clip 10 wedges itself in the top of the bicipital groove 14, as seen in FIGS. 8-10, which will be discussed further below. FIGS. 6 and 7 show a further grasper technique where the biceps tendon 12 is grasped, the clip 10 secured thereto and then deployed to be wedged in the bicipital groove 14.

In sum, the clip 10 is deployed preferably through an accessory posterior or superior arthroscopic portal (not shown). The biceps tendon 12 is preferably pulled into the joint 46 through the standard portal and an inserter instrument 50, the arms of which are shown in broken lines in FIG. 3, would be placed through an accessory portal. Such inserters 50 are so well known in the art that they need not be discussed herein in further detail. The clip 10 is advanced until the biceps tendon 12 is grasped completely at the location where the tendon exits the joint 46 in the at rest position. The inserter 50 is squeezed to deploy the device 10 to secure it to the tendon 12. Referring back to FIGS. 1-3, the arms or fingers of the inserter 50 are located in recesses 48a, 48b in the outer surfaces of the two sides 22a, 22b of the clip 10 to optimize the closing force of the inserter 50 to ensure that the clip 10 is successfully closed to the appropriate depth according to the thickness and configuration of the biceps tendon 12. The proximal portion of the tendon 12 is then resected.

As noted above, if sutures (not shown) were needed these would be placed prior to the deployment of the device 10 using holes 20. The sutures would then be shuttled through the soft tissue or placed in a knotless anchor for fixation to the bone. Such sutures are preferably employed as a secondary/supplemental anchoring of the biceps tendon 12 compared to the primary anchoring using the clip 10 of the present invention.

FIGS. 8-10 show the results of the resultant deployment of the clip 10 into the joint 46 and the wedging thereof in the joint 46 in accordance with the present invention. FIG. 8 shows a close-up view of a deployed clip 10 of the present invention where it is wedged internally in the bicipital groove 14 with the subscapularis 52 and supraspinatus 54 on opposing sides thereof with interval 58. Most importantly, since the clip 10 is larger than the size of the bicipital groove 14, the biceps tendon 12 attached thereto cannot migrate back down into the bicipital groove 14. FIG. 9 shows the clip 10 of the present invention deployed in the bicipital groove 14 as viewed from the outside of the joint 46 while FIG. 10 shows the clip 10 of the present invention deployed in the bicipital groove 14 as viewed from inside the joint 46.

The clip device 10 of the present invention is made of a material that is biologically compatible with human tissue. It is preferably larger than the long head of the biceps tendon 12 so that it can circumferentially grasp the tendon 12. It may or may not contain ridges or teeth 16 to help grasp the tendon 12. It has a locking mechanism at one end 26a, 26b and a hinge 60 at the opposing end. The locking mechanism 26a, 26b is preferably a ratchet but may be other configurations and structures. The hinge 60 is preferably an angled portion of the clip 10 that provides enough flexure so that the opposing sides 22a, 22b of the clip 10 can be moved relative to each other. It is also possible, although not shown, that other structures may be used to provide the desired hinging action, such as a reduction of the thickness of the clip 10 in the desired hinge area.

The clip 10 of the present invention preferably has a wedge type shape so it can wedge itself in the bicipital groove 14 of the humerus that separates the greater tubercle from the lesser tubercle, as best seen in FIG. 8. As stated above, the suture holes 20 allow a secondary fixation of the grasped tendon 12 that allow it to then be secured to the bone, the head of which is 32. Any suitable instrument may be used to deploy the clip 10 arthroscopically as desired in accordance with the present invention (not shown). As can be understood, because most of such biceps tenodesis procedures are arthroscopic, it would require a long shaft between the grip and the jaws/arms. Such instruments are so well known that they need now be discussed in further detail herein.

In summary, the clip 10 of the present invention makes it easier to grasp the tendon 12. Because it is only one device, fewer steps are involved it placing it into position. In many cases, anchoring of the biceps tendon 12 with the clip 10 of the present invention alone, further secondary suturing is not needed. Thus, it is easier to place the clip 10 in the proper position because the tendon 12 can be manipulated with another instrument while the clip 10 is being secured to the biceps tendon 12. Thus, there is typically no need for a complicated suture to be passed.

After deployment of the clip 10, it typically needs to hold the tendon 12 for about 8-10 weeks for the tendon 12 to fix itself to the bone. If the clip 10 of the present invention is made of a dissolvable material, which is envisioned as an option herein, it could then be absorbed away. If it is made of a durable material it would lose its function as a restraint. Therefore, it is preferred that it is incorporated in the scar that is tenodesing the biceps tendon 12.

Turning back to the possible need for suturing, the suture holes 20 discussed above may also be provided to allow it to have a secondary fashion of fixation in case it is used in either open situations or if there is a compromise of a portion of the restraints present at the entrance to the bicipital groove 14. Because the clip 10 uses its increased dimension to prevent the biceps tendon 12 from retracting down the groove 14, any compromise of the soft tissue restraints to the sides and top of the bicipital groove 14 may need secondary fixation. This is well understood in medical surgery as a so-called “belt and suspender” configuration for improved results and reliability of the surgical procedure.

The clip device 10 of the present invention is easy and inexpensive to manufacture. For example, the clip 10 of the present invention may be 3D printed or injection molded as very low cost and could be made of various materials that are biocompatible and have been used in the manufacturing of medical devices.

It would be appreciated by those skilled in the art that various changes and modifications can be made to the illustrated embodiments without departing from the spirit of the present invention. All such modifications and changes are intended to be covered by the appended claims.

What is claimed is:

1. A proximal biceps clip for a biceps tendon, comprising:
a first portion and a second portion; first ends of the first portion and the second portion being hingedly connected together; second ends of the first portion and the second portion being locked to each other;
a capture region defined by inner surfaces of the first portion and the second portion; a grip region defined by outer surfaces of the first portion and the second portion;
the proximal biceps clip being larger than a bicipital groove of a humeral head;
wherein a tendon captured between the first portion and the second portion locked together secures the tendon in place by wedging the biceps clip into the bicipital groove preventing the proximal tendon from retracting down into the bicipital groove as it is larger than the clip device.

2. The proximal biceps clip of claim 1, wherein teeth or ridges are provided on the inner surface of the first portion and the second portion at the groove to improve grasping of the biceps tendon.

3. The proximal biceps clip of claim 1, wherein the respective second ends of the first portion and the second portion are locked to each other using a ratchet mechanism.

4. The proximal biceps clip of claim 1, wherein the second ends of the first portion and the second ends are releasably locked to each other.

5. The proximal biceps clip of claim 1, wherein the outer surfaces of the first portion and the second portion each define a recess configured and arranged for locating a gripping instrument to facilitate closure and locking of the clip on the biceps tendon.

6. The proximal biceps clip of claim 1, wherein the proximal biceps clip is made of biocompatible material.

7. The proximal biceps clip of claim 1, wherein the proximal biceps clip is wedged inside the joint.

8. The proximal biceps clip of claim 1, wherein the proximal biceps clip is deployed arthroscopically.

9. The proximal biceps clip of claim 1, further comprising holes for suturing the clip for a secondary fixation of the biceps tendon and clip in place.

10. A method for deploying a proximal biceps clip for a biceps tendon, comprising the steps of:
providing a clip having a first portion and a second portion; first ends of the first portion and the second portion being hingedly connected together; second ends of the first portion and the second portion being locked to each other; a capture region being defined by inner surfaces of the first portion and the second portion; a grip region defined by outer surfaces of the first portion and the second portion; the proximal biceps clip being larger than a bicipital groove of a humeral head;
arthroscopically pulling the biceps tendon out through the joint;
securing the clip to the biceps tendon in locked fashion whereby the biceps tendon is captured between the first portion and the second portion locked together;
releasing the biceps tendon with clip locked thereto to permit it to partially retract back into the bicipital groove;
wedging the biceps clip into the bicipital groove thereby preventing the proximal tendon from further retracting down into the bicipital groove because it is larger than the clip device.

11. The method of claim 10, wherein teeth or ridges are provided on the inner surface of the first portion and the second portion at the groove to improve grasping of the biceps tendon.

12. The method of claim 10, wherein the respective second ends of the first portion and the second portion are locked to each ratcheting.

13. The method of claim 10, wherein the second ends of the first portion and the second ends are releasably locked to each other.

14. The method of claim 10, wherein the outer surfaces of the first portion and the second portion each define a recess configured and arranged for locating a gripping instrument to facilitate closure and locking of the clip on the biceps tendon.

15. The method of claim 10, wherein the proximal biceps clip is made of biocompatible material.

16. The method of claim 10, further comprising the step of wedging the proximal biceps clip inside the joint at the bicipital groove.

17. The method of claim 10, wherein the proximal biceps clip is deployed arthroscopically.

18. The method of claim 10, further comprising the step of:
providing suture holes in the clip; and
suturing the biceps tendon and clip in place for a secondary fixation.
